(19) United States
(12) Patent Application Publication
(10) Pub. No.: US 2025/0331704 A1
(43) Pub. Date: Oct. 30, 2025

(54) MEDICAL DEVICE WIRE ACTUATION COMPONENTS, ASSEMBLIES, AND ASSOCIATED METHODS

(71) Applicant: Boston Scientific Scimed Inc., Maple Grove, MN (US)

(72) Inventors: Sean POWELL, Holden, MA (US); Matthew Robert JAGELSKI, Milford, MA (US)

(73) Assignee: Boston Scientific Scimed Inc., Maple Grove, MN (US)

(21) Appl. No.: 19/187,490

(22) Filed: Apr. 23, 2025

Related U.S. Application Data
(60) Provisional application No. 63/638,970, filed on Apr. 26, 2024.

(51) Int. Cl. A61B 1/005; A61B 1/00; A61B 1/008

(52) U.S. Cl. CPC A61B 1/0055; A61B 1/00098; A61B 1/00128; A61B 1/008

(57) ABSTRACT

An articulation portion of a medical device may include a plurality of links arranged longitudinally about an axis. Each link may include a center lumen and at least one lumen extending longitudinally through a wall of each respective link, which may be configured to receive an articulation wire. A distalmost link of the plurality of links may include a first cutout on first radial surface of the distalmost link, a second cutout on a second radial surface of the distalmost, and a third cutout on the first radial surface of the distalmost link. The first radial surface may be one of a radially outer surface or a radially inner surface, and the second radial surface may be another of the radially outer surface or the radially inner surface. The articulation wire may extend through each of the first cutout, the second cutout, and the third cutout.

### CROSS-REFERENCE TO RELATED APPLICATIONS

This application claims the benefit of priority to U.S. Provisional Application No. 63/638,970, filed on Apr. 26, 2024, which is incorporated by reference herein in its entirety.

### TECHNICAL FIELD

Various aspects of this disclosure relate generally to medical device wire actuation components, assemblies, and associated methods. In particular, aspects of this disclosure pertain to various configurations of actuating wires for a shaft or a distal tip of a medical device, among other aspects.

### BACKGROUND

Medical scope devices, such as duodenoscopes, may include a handle and a shaft, and the shaft may be insertable into a body lumen of a subject. The handle may include one or more actuators, including buttons, knobs, levers, etc., to control aspects of the medical device via one or more actuating wires coupled to the actuator(s). For example, the actuating wire(s) may be configured to control articulation of a distal portion of the shaft and/or movement of an elevator disposed at a distal tip fixed to the distal end of the shaft. A proximal end of the actuating wire(s) may be coupled to the actuator(s) to control articulation of a distal portion of the shaft and/or movement of the elevator disposed at a distal tip. The actuating wire(s) may extend through the shaft. The actuating wire(s) that control articulation of the distal portion of the shaft may extend through the shaft and terminate at a distal end of the shaft. The actuating wire(s) that control movement of the elevator may terminate at the distal tip (e.g., at the elevator).

### SUMMARY

Each of the aspects disclosed herein may include one or more of the features described in connection with any of the other disclosed aspects.

In an example, an articulation portion of a medical device may include a plurality of links arranged longitudinally about an axis. Each link may include a center lumen and at least one lumen extending longitudinally through a wall of each respective link. The at least one lumen of each link may be configured to receive an articulation wire. A distalmost link of the plurality of links may include a first cutout on first radial surface of the distalmost link, a second cutout on a second radial surface of the distalmost, and a third cutout on the first radial surface of the distalmost link. The first radial surface may be one of a radially outer surface or a radially inner surface, and the second radial surface may be another of the radially outer surface or the radially inner surface. The articulation wire may extend through each of the first cutout, the second cutout, and the third cutout.

Any of the devices disclosed herein may include any of the following features, additionally or alternatively, in any combination.

The first cutout may be configured to impart a first bend on the articulation wire. The third cutout may be configured to impart a second bend on the articulation wire. The second cutout may be configured to impart a third bend on the articulation wire. Each of the first bend, the second bend, and the third bend may be approximately 180 degrees. Each of the first cutout and the second cutout may extend only partially through a wall of the distalmost link.

The distalmost link may further include a first articulation wire lumen extending longitudinally through a wall defining the distalmost link. The first articulation wire lumen may be in fluid connection with the first cutout. The articulation wire may extend through the first articulation wire lumen. The distalmost link may further include a second articulation wire lumen extending longitudinally through a wall defining the distalmost link. The second articulation wire lumen may be in fluid connection with the second cutout. The articulation wire may extend through the second articulation wire lumen.

A proximal opening of each of the first articulation wire lumen and the second articulation wire lumen may be disposed on a proximal surface of the distalmost link. A distal opening of each of the first articulation wire lumen and the second articulation wire lumen may be disposed on a proximal surface of the first cutout and the second cutout, respectively. The third cutout may extend only partially through a wall of the distalmost link. The articulation wire may extend from the first cutout to the third cutout via a first opening defined therebetween. The articulation wire may extend from the third cutout to the second cutout via a second opening defined therebetween.

The third cutout may include a protrusion. The articulation wire may loop around the protrusion. The articulation wire may overlap itself on a proximal side of the protrusion.

In another example, an articulation portion of a medical device may include a plurality of links arranged longitudinally about an axis. Each link may include a center lumen and at least one lumen extending longitudinally through a wall of each respective link. The at least one lumen of each link may be configured to receive an articulation wire. A distalmost link of the plurality of links may include a channel having a first window disposed on a radially outer surface of the distalmost link and a second window disposed on a radially outer surface of the distalmost link. A curve extending circumferentially about the outer surface of the distalmost link may extend longitudinally through each of the first window and the second window. The articulation wire may extend through each of the first window and the second window of the channel.

Any of the devices disclosed herein may include any of the following features, additionally or alternatively, in any combination. The channel may include a third window disposed on a radially inner surface of the distalmost link. At least a portion of the third window may be proximal of and parallel to the first window and the second window.

In a further example, an elevator of a medical device may include a body and a coupler configured to receive an elevator wire. The coupler may extend laterally from a surface of the body. The elevator wire may be configured to loop around the coupler.

Any of the devices disclosed herein may include any of the following features, additionally or alternatively, in any combination. The coupler may include a cavity having an opening disposed on a front face of the coupler. The cavity may be configured to receive a distal end of the elevator wire. The coupler may include a groove extending around at least a portion of the coupler. The groove may receive the elevator wire.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects this disclosure and, together with the description, serve to explain the principles of the disclosure.

FIGS. 1A and 1B depict an exemplary medical device, according to aspects of this disclosure.

FIG. 2A depicts an exemplary articulation portion of the medical device, according to aspects of this disclosure.

FIGS. 2B-2D depict various views of a distalmost link of the articulation portion, according to aspects of this disclosure.

FIG. 3 depicts an alternative exemplary distalmost link, according to aspects of this disclosure.

FIGS. 4A and 4B depict an alternative exemplary distal link of the articulation portion, according to aspects of this disclosure.

FIGS. 5A and 5B depict an alternative exemplary distal link of the articulation portion, according to aspects of this disclosure.

FIG. 6 depicts an alternative exemplary distal link of the articulation portion, according to aspects of this disclosure.

FIGS. 7A-7B, 8A-8B, 9A-9B, and 10A-10B illustrate alternative exemplary distal links of the articulation portion, according to aspects of this disclosure.

FIG. 11 depicts an exemplary elevator, according to aspects of this disclosure.

FIG. 12 depicts an alternative exemplary elevator, according to aspects of this disclosure.

### DETAILED DESCRIPTION

A duodenoscope or other medical device (e.g., an endoscopic ultrasonography (“EUS”) scope, gastroscope, endoscope, or colonoscope) may include an insertion portion comprised of a flexible shaft, an articulation portion, and a distal tip. The articulation portion may be coupled to a distal end of the shaft. The distal tip may be coupled to a distal end of the articulation portion. The articulation portion may be deflected, or articulated, via one or more actuating wires or cables operatively coupled to a first actuator in a handle of the duodenoscope. The actuating wire(s) may extend longitudinally through a length of the shaft and terminate at an articulation portion of the shaft. For example, when an operator activates the first actuator, the articulation portion may be deflected in one or more directions.

The distal tip may include an elevator. The elevator may be operatively connected to a second actuator in a handle of the duodenoscope, e.g., via different actuating wire(s) or cable(s). The elevator actuation wire(s) may extend longitudinally through a length of the shaft and the articulation portion and terminate at the elevator. When an operator activates the second actuator, the elevator may be raised/lowered via the actuation wire(s). When an operator inserts an accessory device (e.g., an instrument for guidewire) into a working channel of the duodenoscope and advances the instrument through the working channel, the elevator originally may be in a lowered configuration. After the accessory device has been extended out of a distal opening of the working channel, the elevator may be raised so as to deflect a distal tip of the instrument. For example, such deflection of the elevator may be utilized in conjunction with or independent of the deflection of the articulation portion, e.g., to allow an operator to access a subject's biliary tract and/or to cannulate a subject's major papilla.

A distal end of conventional actuation wire(s) may include a termination member (e.g., a ferrule, a laser welded ball, a crimp, etc.) to assist with keeping a relative position of each wire within the insertion portion. For example, a distal portion of each of the actuation wire(s) configured to deflect the articulation portion may extend at least partially through a longitudinal length of the articulation portion. The termination member may be fixed to a distal end of each actuation wire. The termination member may fixedly couple the distal end of the actuation wire to the articulation portion and prevent proximal movement of the actuation wire(s), e.g., proximally through the articulation portion when the articulation portion is being deflected. Similarly, a distal end of the actuation wire(s) configured to raise/lower the elevator may include a termination member (e.g., a ferrule, a laser welded ball, a crimp, etc.). The termination member may fixedly couple the distal end of the articulation wire to the elevator and prevent proximal movement of the actuation wire(s), e.g., proximally of the elevator when the elevator is being raised/lowered.

The termination members of each actuation wire may occupy valuable space within the distal portion of the insertion portion. Routing each actuation wire within the articulation portion and/or around the elevator may reduce or eliminate a need for the termination member. Reducing or eliminating the need for a termination member on each actuating wire may reduce a number of components for manufacturing the medical device, thereby reducing manufacturing costs and time. Additionally or alternatively, without the termination member fixed to the distal end of each actuating wire, additional space within the distal tip may result in the distal tip having a smaller overall outer diameter and/or a larger inner diameter. The larger inner diameter may accommodate larger components (e.g., larger lumens, electronic components, etc.), for example, without increasing an overall outer diameter of the distal tip or articulation portion.

For ease of description, the one or more actuation wires/cables configured to articulate/deflect the articulation portion may be referred to herein collectively as “articulation wire(s),” and the one or more actuation wires/cables configured to raise/lower the elevator may be referred to herein as “elevator wire(s)”. Unless otherwise noted, the articulation wire(s) and elevator wire(s) may have any or all of the same characteristics as one another. For example, the articulation wire(s) and elevator wire(s) may be comprised of a same material, have the same dimension(s), and/or have a same or similar tensile strength.

Throughout various figures, the arrows “P” and “D” depict proximal and distal directions. Any of the devices disclosed herein may include any of the following features, additionally or alternatively, in any combination. Accordingly, between different embodiments, like numbers will be used to refer to like features, with “100” added to each numeral.

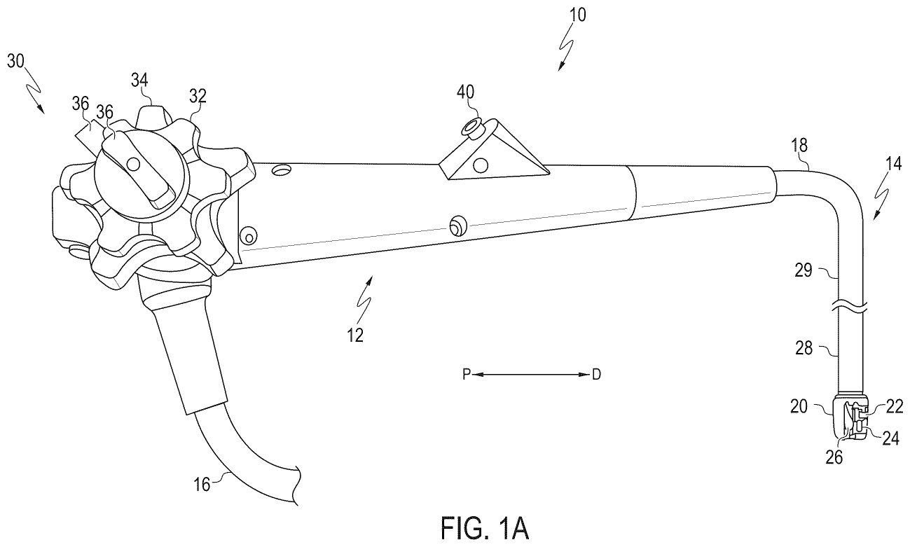

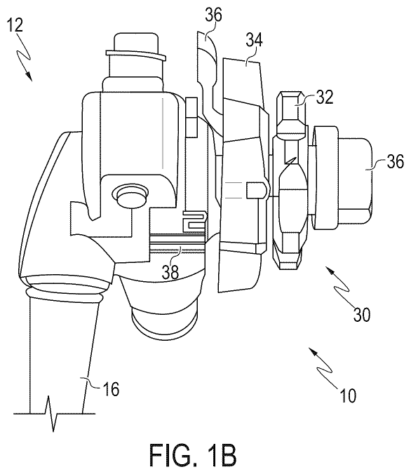

FIG. 1A depicts an exemplary duodenoscope 10 (or other medical device) having a handle 12 and an insertion portion 14. FIG. 1B shows a proximal end of handle 12. Duodenoscope 10 may also include an umbilicus 16 for purposes of connecting duodenoscope 10 to sources of, for example, air, water, suction, power, etc., as well as to image processing and/or viewing equipment. Although a duodenoscope may be referenced herein, it will be appreciated that the disclosure also encompasses endoscopes, bronchoscopes, gastroscopes, EUS scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

Insertion portion 14 may include a shaft 18, an articulation portion 28, and a distal tip 20. Distal tip 20 may include an imaging device 22 (e.g., a camera) and a lighting source 24 (e.g., an LED or an optical fiber). Distal tip 20 may be side-facing. That is, imaging device 22 and lighting source 24 may face radially outward, perpendicularly, approximately perpendicularly, or otherwise transverse to a longitudinal axis of shaft 18 and distal tip 20. Alternatively, imaging device 22 and lighting source 24 may face distally (be forward-facing). Additionally or alternatively, distal tip 20 may include one or more imaging devices 22 that face in more than one direction. For example, a first imaging device 22 may face radially outward, and a second imaging device 22 may face distally (e.g., approximately parallel to a longitudinal axis of distal tip 20/shaft 18).

Distal tip 20 may also include an elevator 26 for changing an orientation of a tool inserted in a working channel of duodenoscope 10. Elevator 26 may alternatively be referred to as a swing stand, pivot stand, raising base, or any other suitable term. Elevator 26 may be pivotable, e.g., via one or more elevator wire(s) that extends from handle 12, through shaft 18 and articulation portion 28, to elevator 26.

An outer sheath 29 may extend an entire length of shaft 18. In aspects, outer sheath 29 may also extend over articulation portion 28. Outer sheath 29 may be comprised of one continuous sheath that extends over shaft 18 and articulation portion 28. In other aspects, outer sheath 29 may be comprised of two or more sheaths having a discrete length, such that, for example, a first sheath extends along a length of shaft 18 and/or a second sheath extends along articulation portion 28.

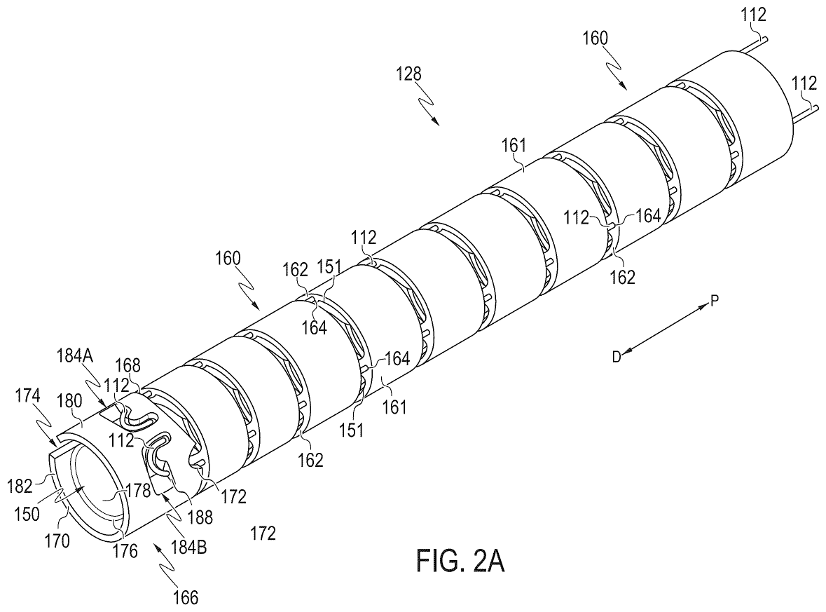

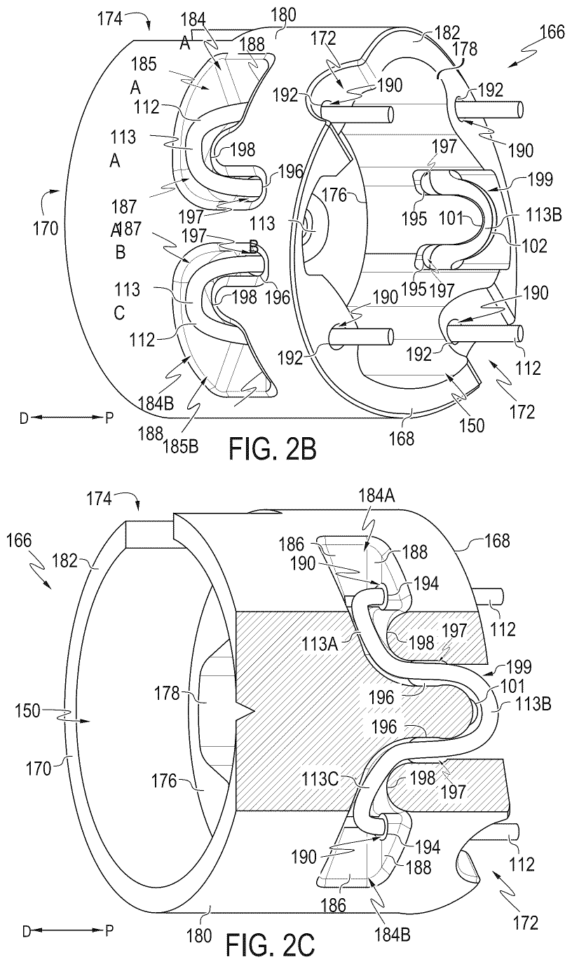

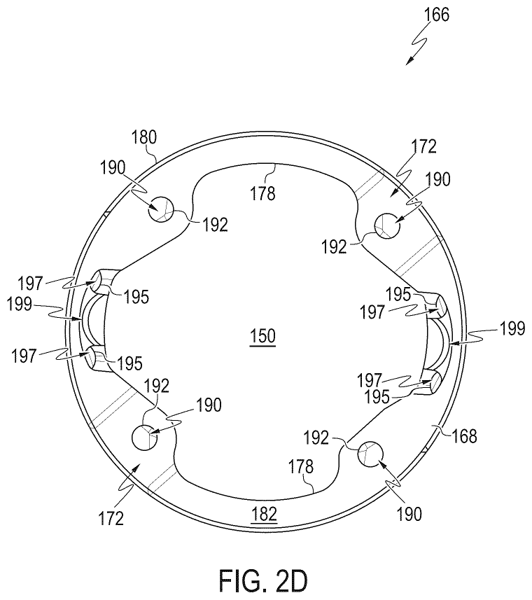

In aspects, articulation portion 28 may be fixed (directly or indirectly) to a distal end of shaft 18 and a proximal end of distal tip 20. Articulation portion 28 may be comprised of, for example, one or more articulation joints (e.g., see FIG. 2A). Shaft 18 and articulation portion 28 may include a variety of structures, which are known or may become known in the art. One or more articulation wires (e.g., see FIG. 2A) may extend from handle 12, through shaft 18, to a distal end of articulation portion 28. The articulation wire(s) may enable deflection of articulation portion 28 in one or more directions (e.g., up, down, left, right, etc.).

Referring still to FIGS. 1A and 1B, handle 12 may have one or more actuators/control mechanisms 30. Control mechanisms 30 may provide control of articulation portion 28 and/or may allow for provision of air, water, suction, etc. For example, handle 12 may include control knobs 32, 34 to enable deflection of articulation portion 28 in one or more directions. For example, one of knobs 32, 34 may provide left/right control of articulation portion 28, and the other of knobs 32, 34 may provide up/down control of articulation portion 28. Handle 12 may further include one or more locking mechanisms 36 (e.g., knobs or levers) for preventing steering and/or braking of articulation portion 28 in at least one of an up, down, left, or right direction. Handle 12 may include an elevator control lever 38 (see FIG. 1B). Elevator control lever 38 may raise and/or lower elevator 26, via connection between lever 38 and elevator wire(s) that extends from lever 38, through shaft 18, to elevator 26. A port 40 of handle 12 may allow passage of a tool through port 40, into a working channel of the duodenoscope 10, through shaft 18 and articulation portion 28, and to distal tip 20.

In use, an operator may insert at least a portion of insertion portion 14 into a body lumen of a subject. Distal tip 20 may be navigated to a procedure site in the body lumen. Articulation portion 28 may be deflected to assist in accessing the procedure site within the subject. The operator may insert an accessory device (not shown) into port 40, and pass the accessory device through insertion portion 14 via a working channel to distal tip 20. The accessory device may exit the working channel at distal tip 20. The user may use elevator control lever 38 to raise elevator 26 and angle the accessory device toward a desired location (e.g., a papilla of the pancreatic-biliary tract). The user may use the accessory device to perform a medical procedure.

FIG. 2A depicts an exemplary articulation joint assembly 128. Articulation portion 28 of FIG. 1A may include articulation joint assembly 128. For example, articulation joint assembly 128 is shown in FIG. 2A without outer sheath 29 of FIG. 1A. Although features may be described with respect to a particular example herein, it will be appreciated that the features described herein may be combined in any suitable combination. Articulation joint assembly 128 described herein may be used in conjunction with duodenoscope 10 or another medical device. For convenience, features of duodenoscope 10 will be referenced below but will not be understood to limit features of articulation joint assembly 128, described herein.

Articulation joint assembly 128 may be comprised of one or more links 160 and a distalmost link 166 arranged along a longitudinal axis of articulation joint assembly 128. Distalmost link 166 may define a distalmost portion of articulation joint assembly 128. Link(s) 160 may be arranged proximally of distalmost link 166. FIGS. 2B-2D illustrate various views of distalmost link 166 and may be referred to in conjunction with FIG. 2A. For example, FIG. 2B illustrates a perspective view of distalmost link 166; FIG. 2C illustrates a partial cross-sectional view of distalmost link 166; and FIG. 2D illustrates a back view of distalmost link 166 (i.e., a view facing in a distal direction).

Articulation joint assembly 128 (e.g., link(s) 160 and distalmost link 166) may define a central lumen 150 extending longitudinally through each of links 160 and distalmost link 166. Central lumen 150 may be capable of receiving sheaths, tools, imaging devices, cables, wires, and/or other devices or components associated with duodenoscope 10, e.g., to perform endoscopic or other medical procedures. Central lumen 150 of articulation joint assembly 128 may be in fluid communication with one or more lumens of shaft 18. It will be understood that insertion portion 14, including shaft 18, articulation portion 28, and articulation joint assembly 128, are not limited to a single central lumen 150, and may include any number of lumens necessary for performing procedures.

Central lumen 150 of each of link(s) 160 may be defined by an inner surface 151. A wall 162 between inner surface 151 and an outer surface 161 of each link 160 may include one or more articulation wire lumens 164. Articulation wire lumen(s) 164 of each link of the plurality of links 160 may extend through wall 162, e.g., parallel to the longitudinal axis of articulation joint assembly 128. Articulation wire(s) 112 may extend distally from handle 12 duodenoscope 10, through shaft 18, and through articulation wire lumen(s) 164 of each link 160. In some aspects, each link 160 may have one, two, three, four, or more articulation wire lumen(s) 164, e.g., spaced evenly or unevenly around central lumen 150.

Referring to FIGS. 2A-2D, distalmost link 166 may have a proximal face 168 and a distal face 170. Proximal face 168 may have any number of proximal features 172 (e.g., notches, protrusions, etc.) or angled surfaces to facilitate a connection between distalmost link 166 and link(s) 160. Similarly, distal face 170 may have any number of distal features 174 (e.g., notches, protrusions, etc.) or angled surfaces to facilitate a connection between distalmost link 166 and distal tip 20 (see FIG. 1A). In some aspects, distalmost link 166 may have a recessed wall or surface 176, which may be recessed relative to distal face 170. Recessed surface 176 may extend radially inward from an outer wall of distalmost link 166, proximally of distal face 170. Recessed surface 176 may form a shelf or similar structure. A portion of distal tip 20 may be received by distalmost link 166 and abut recessed surface 176.

Distalmost link 166 may also have an inner surface 178 and an outer surface 180 defining a wall 182. Outer surface 180 may be a radially outer surface of distalmost link 166. Central lumen 150 may be defined by inner surface 178. Inner surface 178 may be a radially inner surface of distalmost link 166. Outer surface 180 may include a first cutout 184A and a second cutout 184B, each of which may extend at least partially radially inward into wall 182 of distalmost link 166. For example, cutouts 184A and 184B may form recesses. In aspects, cutouts 184A and 184B may extend through an entire thickness of wall 182, to and/or through inner surface 178. Although not shown, distalmost link 166 may have additional cutouts (e.g., one, two, three, or more) on a side of distalmost link 166 that faces into the page in FIGS. 2A-2C. Each of the additional cutouts may have any or all of the features of cutouts 184A, 184B, described herein. Cutouts 184A, 184B may each be mirror images of one another, and, thus, may each have the same features. In these aspects, element numbers for features of first cutout 184A may be used to identify similar element numbers for features of second cutout 184B. For example, cutouts 184A, 184B may each have a radially inner surface 186 (e.g., a back surface) (FIG. 2C) and a proximal surface 188 (FIGS. 2B and 2C). Proximal surface 188 of each cutout 184A, 184B may be perpendicular to the longitudinal axis of distalmost link 166.

One or more articulation wire lumens 190 may extend through wall 182 of distalmost link 166, e.g., parallel to the longitudinal axis of articulation joint assembly 128. For example, respective proximal openings 192 of articulation wire lumen(s) 190 may be disposed on proximal face 168 of distalmost link 166, as shown in FIGS. 2B and 2D. In aspects, one or more proximal openings 192 may be disposed within or between proximal features 172. Articulation wire lumen(s) 190 of distalmost link 166 may be similarly sized and shaped as articulation wire lumen(s) 164 of link(s) 160. In these aspects, articulation wire lumen(s) 190 of distalmost link 166 may be aligned with articulation wire lumen(s) 164 of link(s) 160 such that articulation wire(s) 112 may extend through the articulation wire lumen(s) 164 and articulation wire lumen(s) 190. Respective distal openings 194 of articulation wire lumen(s) 190 may be disposed on proximal surface 188 of each cutout 184A, 184B, as shown in FIG. 2C.

Articulation wire(s) 112 may be a single wire (or group of wires or cable) that extends through both cutouts 184A, 184B. For example, a portion of articulation wire(s) 112 may extend through a first of articulation wire lumen(s) 190 of distalmost link 166 and into first cutout 184A (e.g., via distal opening 194 on proximal surface 188 of first cutout 184A). A first bend 113A of articulation wire(s) 112 may then be formed such that, for example, articulation wire(s) 112 is/are bent approximately 180 degrees. First cutout 184A may have a curved surface 198 at a proximal side of first cutout 184A, e.g., to assist in forming first bend 113A of articulation wire(s) 112.

After first bend 113A, articulation wire(s) 112 may extend radially inward (e.g., towards central lumen 150) and through an opening 196 disposed on radially inner surface 186 of first cutout 184A. Opening 196 may be closer to second cutout 184B than opening 194 is (in other words, opening 194 may be further from second cutout 184B than opening 196 is). Opening 196 of first cutout 184A may be a distal opening of a lumen 197. Lumen 197 may extend through an entirety of wall 182 of distalmost link 166. Lumen 197 may have a proximal opening 195 on inner surface 178.

As most clearly shown in FIG. 2B, inner surface 178 of distalmost link 166 may include a recess, channel, or cutout 199. Cutout 199 may have a radially open inner side, such that cutout 199 may be a recess formed in inner surface 178. Cutout 199 may extend along innermost surface 178, for example, between opening 195 of lumen 197 of first cutout 184A and opening 195 of lumen 197 of second cutout 184B. In these aspects, articulation wire(s) 112 may extend from first cutout 184A into second cutout 184B, via cutout 199. Cutout 199 may have a curved, U-shaped surface. For example, cutout 199 may have a curved surface 101 to assist in forming a second bend 113B of articulation wire(s) 112. Curved surface 101 may extend proximally, such that a central portion of curved surface 101 is proximal of sides of curved surface 101. Second bend 113B may be approximately 180 degrees. A proximal end of cutout 199 may be a proximalmost end of distalmost link 166, such that cutout 199 has an open proximal side. Cutout 199 may have a surface 102 that is recessed radially outward relative to adjacent portions of inner surface 178. However, cutout 199 may extend through only a portion of a thickness of wall 182, such that cutout 199 is not open on outer surface 180.

Articulation wire(s) 112 may extend radially outward (e.g., away from central lumen 150) through opening 195 of lumen 197 of second cutout 184B and extend into second cutout 184B through opening 196. In these aspects, articulation wire(s) 112 may have a third bend 113C. Third bend 113C of articulation wire(s) 112 may bend articulation wire(s) 112 approximately 180 degrees. Articulation wire(s) 112 may then extend through opening 194 disposed on proximal surface 188 of second cutout 184B and through articulation wire lumen 190 of distalmost link 166. Articulation wire(s) 112 may then extend proximally through articulation lumens 164 of one or more links 160, and through shaft 18. In aspects, a single articulation wire 112 (or group of wires such as a cable) may extend through each of first cutout 184A and second cutout 184B. The single articulation wire 112 may control the deflection of articulation portion 28 in two or more directions.

The three bends (e.g., first bend 113A, second bend 113B, and third bend 113C) of articulation wire(s) 112 may secure articulation wire(s) 112 within distalmost link 166. In these aspects, the three bends 113A, 113B, 113C of articulation wire(s) 112 may create a capstan effect. For example, a tensile force that may be applied to articulation wire(s) 112 (e.g., during articulation) may increase exponentially due to the three bends 113A, 113B, 113C without articulation wire 112 being removed from distalmost link 166. In these aspects, the three bends 113A, 113B, 113C may assist in preventing articulation wire(s) 112 from being pulled proximally out of distalmost link 166.

Cutouts 184A, 184B may have open radially outer sides, such that inner surface 186 is visible from an outside of distalmost link 166. In aspects, cutouts 184A, 184B may form recessed cavities within wall 182. Shown clearly in FIG. 2B, cutouts 184A and 184B may each have a first portion 185A, 185B, respectively, and a second portion 187A, 187B, respectively. First portions 185A, 185B may have an approximately rectangular perimeter. Second portions 187A, 187B may have elongated, channel shapes. Curved surface 198 may be between first portion 185A, 185B and second portion 187A, 187B. Second portions 187A, 187B may extend further in a proximal direction that first portions 185A, 185B. A proximal edge of second portions 187A, 187B may be distal of a proximalmost end of distalmost link 166. Second portion 187A may be closer to second portion 187B than to first portion 185B. Similarly, second portion 187B may be closer to second portion 187A than to first portion 185A. In other words, second portion 187A of first cutout 184A and second portion 187B of second cutout 184B may be adjacent to one another.

Distalmost link 166 may be comprised of a metal (e.g., stainless steel, titanium, etc.) and/or a plastic material (e.g., acrylonitrile butadiene styrene, polycarbonate, etc.). In these aspects, distalmost link 166 may be machined, molded, stamped, or formed via any other suitable means commonly used in the art.

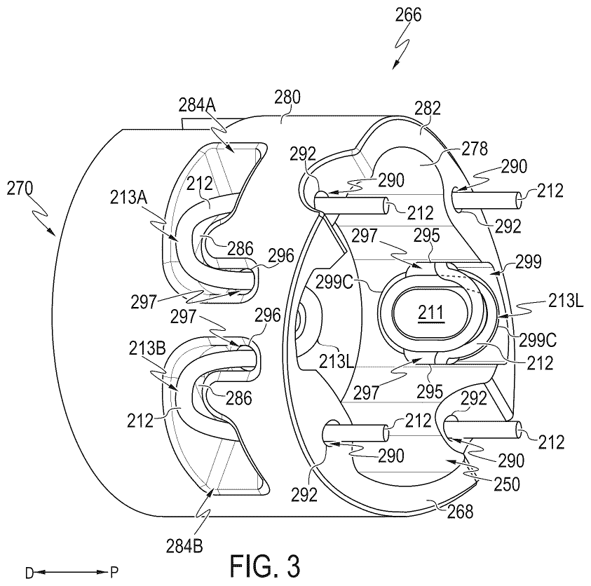

FIG. 3 illustrates an alternative distalmost link 266. For example, FIG. 3 illustrates a perspective view of distalmost link 266. Except as described below, distalmost link 266 may have any or all of the characteristics of distalmost link 166, described above with respect to FIGS. 2A-2D. For example, distalmost link 266 may be arranged along a longitudinal axis of articulation joint assembly 128. Distalmost link 266 may define a distalmost portion of articulation joint assembly 128. Articulation wire(s) 212 may extend through distalmost link 266, as described in further detail below. Articulation wire(s) 212 may have any or all of the characteristics of articulation wire(s) 112, described above.

A central lumen 250 may extend through distalmost link 266. In aspects, central lumen 250 may be defined by an inner surface 278 of distalmost link 266. A wall 282 may be between inner surface 278 and an outer surface 280. Inner surface 278 may define a radially inner surface of distalmost link 266, and outer surface 280 may define a radially outer surface 280 of distalmost link 266. A proximal end of distalmost link 266 may be defined by one or more surfaces 268, and a distal end of distalmost link 266 may be defined by a distal surface 270.

One or more articulation wire lumens 290 may extend longitudinally through wall 282. For example, articulation wire lumen(s) 290 of distalmost link 266 may extend at least partially through wall 282, e.g., parallel to the longitudinal axis of distalmost link 266. Although four articulation wire lumens 290 are shown in FIG. 3, distalmost link 266 may have one, two, three, or more articulation wire lumen(s) 290, e.g., spaced evenly or unevenly around central lumen 250. The one or more articulation wire lumen(s) 290 may be aligned with the articulation wire lumen(s) 164 of links 160. A proximal opening 292 of each articulation wire lumen 290 may be disposed on surface(s) 268.

Outermost surface 280 may include a first cutout 284A and a second cutout 284B, each of which may extend at least partially into wall 282 of distalmost link 166. Cutouts 284A, 284B may each be mirror images of one another, and, thus, may each have the same features. In these aspects, element numbers for features of first cutout 284A may be used to identify similar element numbers for features of second cutout 284B. Cutouts 284A, 284B may have any or all of the same features as cutouts 184A, 184B, previously described. For example, articulation wire 212 may extend into first cutout 284A via a first of articulation wire lumen 290. Articulation wire 212 may be subjected to a first bend 213A within first cutout 284A. Articulation wire 212 may extend radially inward (e.g., towards central lumen 250) through a distal opening 296 disposed on inner wall 286 of first cutout 284A and through a lumen 297. Articulation wire 212 may extend into central lumen 250 from lumen 297 via a proximal opening 295 disposed on inner surface 278. Described in further detail below, second cutout 284B may also include a lumen 297 having a distal opening 296 disposed on inner wall 286 of second cutout 284B and a proximal opening 295 disposed on inner surface 278. In these aspects, proximal opening 295, distal opening 296, and lumen 297 may have any or all of the same characteristics as proximal opening 195, distal opening 196, and lumen 197, described above with respect to FIGS. 2B-2D.

Referring still to FIG. 3, inner surface 278 of distalmost link 266 may include a recessed cutout 299. Cutout 299 may have any of the features of cutout 199, except as specified herein. Cutout 299 may extend along inner surface 278, for example, between first cutout 284A and second cutout 284B. Cutout 299 may include a channel 299C extending around a protrusion 211. For example, channel 299C may be circular or ovular and extend around a perimeter of protrusion 211. In aspects, protrusion 211 may protrude radially inward relative to a recessed surface of channel 299C/cutout 299. Protrusion 211 may have an obround or oval shape.

Articulation wire 212 may extend from proximal opening 296 of first cutout 284A, into cutout 299 (e.g., via lumen 297 of first cutout 284A). Articulation wire 212 may then loop around protrusion 211. For example, articulation wire 212 may first extend around a proximal portion of protrusion 211 and then extend through channel 299C to loop around a distal portion of protrusion 211, before extending proximally back around the proximal portion of protrusion 211. For example, articulation wire 212 may be subjected to a loop 213L (which may be at least 360 degrees) before extending through second opening 296 of second cutout 284B. In aspects, articulation wire 212 may cross over itself along a proximal portion of protrusion 211. Articulation wire 212 may then extend outward (e.g., towards outer surface 280 of distalmost link 266) through lumen 297 (e.g., via proximal opening 295 and distal opening 296 of lumen 297) second cutout 284B. Within second cutout 284B, articulation wire 212 may be subjected to a second bend 213B before extending proximally through a second of articulation wire lumen 290. In some aspects, articulation wire 212 may cross over itself.

Loop 213L may assist in securing articulation wire 212 within distalmost link 266. For example, when articulation wire 212 is pulled proximally, loop 213L may tighten around protrusion 211 and over itself. Thus, in aspects, together with bends 213A, 213B, loop 213L may assist in creating a capstan effect on articulation wire 212.

Distalmost link 266 may be comprised of a metal (e.g., stainless steel, titanium, etc.) and/or a plastic material (e.g., acrylonitrile butadiene styrene, polycarbonate, etc.). In these aspects, distalmost link 266 may be machined, molded, stamped, or formed via any other suitable means commonly used in the art.

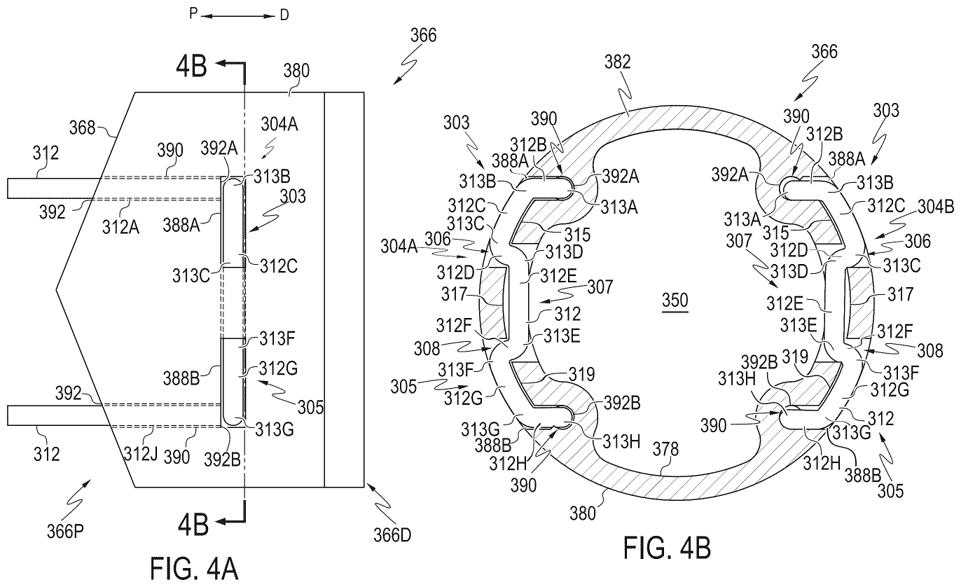

FIGS. 4A and 4B illustrate an alternative distalmost link 366. For example, FIG. 4A illustrates a side view of distalmost link 366; and FIG. 4B illustrates a cross-sectional view of distalmost link 366. Except as described below, distalmost link 366 may have any or all of the characteristics of distalmost link 166 and/or distalmost link 266, described above with respect to FIGS. 2A-2D and 3. For example, distalmost link 366 may be arranged along a longitudinal axis of articulation joint assembly 128. Distalmost link 366 may define a distalmost portion of articulation joint assembly 128. Articulation wire(s) 312 may extend through distalmost link 366, as described in further detail below. Articulation wire(s) 312 may have any or all of the characteristics of articulation wire(s) 112, 212, described above.

A central lumen 350 may extend through distalmost link 366. When assembled with articulation joint assembly 128, central lumen 350 of distalmost link 366 may be aligned with central lumen 150 of link(s) 160. In aspects, central lumen 350 may be defined by an inner surface 378 of distalmost link 366. A wall 382 may be between inner surface 378 and an outer surface 380 of distalmost link 366. Inner surface 378 may define a radially inner surface of distalmost link 366, and outer surface 380 may define a radially outer surface of distalmost link 366. Distalmost link 366 may have a proximal end 366P and a distal end 366D. Proximal end 366P of distalmost link 366 may be defined by one or more surfaces 368. The one or more surfaces 368 may be angled and/or include features to fit with a distal end of proximal link(s) 160.

Wall 382 may include one or more articulation wire lumens 390 extending at least partially through wall 382, e.g., parallel to the longitudinal axis of distalmost link 366. Articulation wire lumen(s) 390 may be aligned with the articulation wire lumen(s) 164 of links 160. A proximal opening 392 of each articulation wire lumen 390 may be disposed on surface(s) 368. Articulation wire lumens 390 are shown in broken lines in FIG. 4B, for example, to illustrate that articulation wire lumens 360 extend longitudinally through wall 382.

As shown in FIG. 4B, a first channel 304A and a second channel 304B may extend through and/or along wall 382 of distalmost link 366. Channels 304A, 304B may be approximately perpendicular to a longitudinal axis of distalmost link 366. First channel 304A may be on a first side of distalmost link 366, and second channel 304B may be on a second side of distalmost link. First channel 304A and second channel 304B may be mirror images of one another, and, thus, may have any or all of the same features. In these aspects, the features of first channel 304A and second channel 304B may have the same element numbers identifying the same features.

Channels 304A, 304B may each have a plurality of segments. For example, channels 304A, 304B may each include a first external cutout 303 (also referred to herein as a first external window) and a second external cutout 305 (also referred to herein as a second external window) on outer surface 380 of distalmost link 366. In aspects, cutouts 303, 305 may be recesses that are recessed with respect to outer surface 380, but may not extend all the way through wall 382.

Channels 304A, 304B may also define an internal cutout 307 (also referred to herein as an internal window) on internal surface 378 of distalmost link 366. Internal cutout 307 may be between external cutout 303 and external cutout 305. In aspects, internal cutout 307 may be a recess that is recessed with respect to inner surface 378, but does not extend all the way through wall 382. When distal link 366 is viewed from the side, each external window 303, 305 and internal window 307 may be linearly aligned with one another. For example, a curve extending circumferentially about outer surface 380 of distal link 366 and perpendicularly to a longitudinal axis of distalmost link 366 may extend through each of external cutouts 303, 305 and internal cutout 307.

An end of external cutout 303 may overlap with a first end of internal cutout 307. In aspects, external cutout 303 may be in communication with internal cutout 307 by a first opening 306 in the overlapped portions of external cutout 303 and internal cutout 307. Similarly, an end of external cutout 305 may overlap with a second end of internal cutout 307. In aspects, external cutout 305 may be in communication with internal cutout 307 by a second opening 308 in the overlapped portions of external cutout 303 and internal cutout 307.

A first distal opening 392A of two articulation wire lumen(s) 390 may be disposed on a first proximal wall 388A of each of first channel 304A and second channel 304B. A second distal opening 392B of two other articulation wire lumens 390 may be disposed on a second proximal surface 388B of each of first channel 304A and second channel 304B.

A single articulation wire 312 (or group of wires such as a cable) may extend through one of channels 304A, 304B. For example, a first segment 312A (FIG. 4A) of articulation wire(s) 312 may extend distally through articulation wire lumen 390 of distalmost link 366. A longitudinal axis of first segment 312A may be approximately parallel to the longitudinal axis of distalmost link 366. After articulation wire 312 extends distally past articulation wire lumen 390 (e.g., via opening 392A disposed on first proximal wall 388A of first channel 304A), articulation wire 312 may extend into first channel 304A. Just distal to distal opening 392A on first proximal wall 388A, articulation wire 312 may be subjected to a first bend 313A (FIG. 4B). First bend 313A may be approximately 90 degrees, e.g., towards outer surface 380 of distalmost link 366, such that a second segment 312B of articulation wire 312 extends away from a longitudinal axis of distal link 366 (e.g., away from central lumen 350). In aspects, second segment 312B may extend along a plane that is approximately perpendicular to first segment 312A. For example, a longitudinal axis of second segment 312B may be approximately perpendicular to the longitudinal axis of first segment 312A.

When second segment 312B of articulation wire 312 reaches first external cutout 303 of first channel 304A, articulation wire 312 may be subjected to a second bend 313B. A third segment 312C of articulation wire 312 may extend along a length of first external cutout 303. Articulation wire 312 may be subjected to a third bend 313C on an opposite end of articulation wire 312 from third segment 312C. A radially inner surface 315 of first cutout 303 may also help to form second bend 313B and third bend 313C of articulation wire 312.

After third bend 313C, a fourth segment 312D of articulation wire 312 may extend through first opening 306 (e.g., by extending towards central lumen 350). On an opposite end of fourth segment 312D, articulation wire 312 may be subjected to a fourth bend 313D. After fourth bend 313D, a fifth segment 312E of articulation wire 312 may extend along a length of internal cutout 307. On an opposite end of fifth segment 312E, articulation wire 312 may be subjected to a fifth bend 313E. A radially outer surface 317 forming internal cutout 307 may also assist in forming fourth bend 313D and fifth bend 313E.

After fifth bend 313E, a sixth segment 312F of articulation wire 312 may extend radially outward through second opening 308, e.g., away from central lumen 350. When sixth segment 312F of articulation wire 312 reaches second external cutout 305, articulation wire 312 may be subjected to a sixth bend 313F. A seventh segment 312G may extend along a length of second external cutout 305. Articulation wire 312 may be subjected to a seventh bend 313G on an opposite end of seventh segment 312G from sixth bend 313F. A radially inner surface 319 forming second external cutout 305 may also assist in forming sixth bend 313F and seventh bend 313G of articulation wire 312.

After seventh bend 313G, an eighth segment 312H may extend radially inward toward central lumen 350. On an opposite end of eighth segment 312H from seventh bend 313G, articulation wire 312 may be subjected to an eighth bend 313H. Eighth bend 313H may be approximately 90 degrees such that a ninth segment 312J (FIG. 4A) of articulation wire 312 extends into and through a second articulation wire lumen 390 of distalmost link 366 (e.g., via opening 392B disposed on second proximal surface 388B of first channel 304A). In these aspects, a longitudinal axis of ninth segment 312J may be approximately perpendicular to a longitudinal axis of eighth segment 312H. The longitudinal axis of ninth segment 312J may be parallel to the longitudinal axis of first segment 312A and the longitudinal axis of distalmost link 366.

In aspects, articulation wire 312 may be further secured to distalmost link 366. For example, an adhesive or epoxy may be applied to articulation wire 312 or distalmost link 366. Additionally or alternatively, articulation wire 312 may be heat staked, swaged, or stamped into distalmost link 366 to further secure articulation wire 312 to distalmost link 366.

Although first channel 304A is referred to above, it will be appreciated that a second articulation wire 312 extending through second channel 304B may follow the same path as described above. In aspects, channels 304A, 304B may be M- or W-shaped such that, for example, articulation wire(s) 312 is/are weaved through each respective channel 304A, 304B.

Each bend (e.g., first bend 313A, second bend 313B, third bend 313C, fourth bend 313D, fifth bend 313E, sixth bend 313F, seventh bend 313G, eighth bend 313H) may be approximately 80-120 degrees. The eight bends of distalmost link 366 may create a capstan effect on articulation wire 312. For example, a tensile force that may be applied to articulation wire 312 (e.g., during articulation) may increase exponentially due to the eight bends of articulation wire 312. In these aspects, the eight bends may assist in preventing articulation wire 312 from being pulled proximally out of distalmost link 366.

Distalmost link 366 may be comprised of a metal (e.g., stainless steel, titanium, etc.) and/or a plastic material (e.g., acrylonitrile butadiene styrene, polycarbonate, etc.). In these aspects, distalmost link 366 may be machined, molded, stamped, or formed via any other suitable means commonly used in the art.

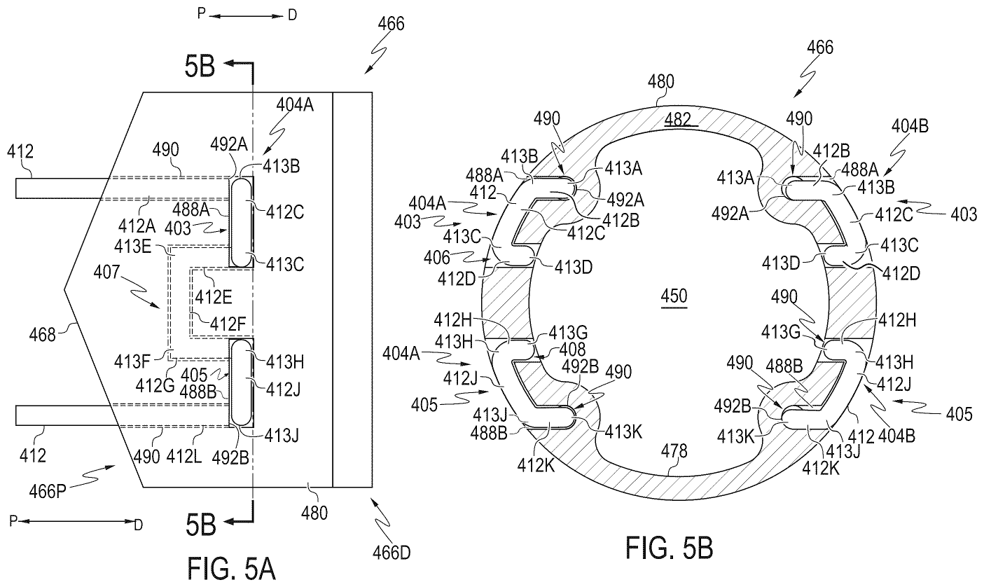

Referring to FIGS. 5A and 5B, an articulation wire 412 may be subjected to additional bends (e.g., nine bends, ten bends, etc.). In some aspects, the additional bends may be in a longitudinal direction (e.g., parallel to the longitudinal axis of a distalmost link). For example, FIGS. 5A and 5B illustrate an alternative distalmost link 466 having additional longitudinal bends. FIG. 5A illustrates a side view of distalmost link 466, and FIG. 5B illustrates a cross-sectional view of distalmost link 466. Distalmost link 466 may have any or all of the characteristics of one or more of distalmost link 166, 266, 366, except as described below. In particular, distalmost link may be similar to distalmost link 366, except as specified below. For example, distalmost link 466 may be arranged along a longitudinal axis of articulation joint assembly 128. Links 160 may be arranged proximally to distalmost link 466. Distalmost link 466 may define a distalmost portion of articulation joint assembly 128. Articulation wire(s) 412 may extend through distalmost link 466, as described in further detail below. Articulation wire(s) 412 may have any or all of the characteristics of articulation wire(s) 112, 212, 312 described above.

A central lumen 450 may extend through distalmost link 466. When assembled with articulation joint assembly 128, central lumen 450 of distalmost link 466 may be aligned with central lumen 150 of link(s) 160. In aspects, central lumen 450 may be defined by an inner surface 478 of distalmost link 466. A wall 482 may be between inner surface 478 and an outer surface 480 of distalmost link 466. Inner surface 478 may define a radially inner surface of distalmost link 466, and outer surface 480 may define a radially outer surface of distalmost link 466. Distalmost link 466 may have a proximal end 466P and a distal end 466D. In some aspects, proximal end 466P may be defined by one or more surfaces 468. The one or more surfaces 468 may be angled.

Wall 482 may include one or more articulation wire lumens 490. Articulation wire lumen(s) 490 of distalmost link 466 may extend at least partially through wall 482, e.g., parallel to the longitudinal axis of distalmost link 466. Although four articulation wire lumens 490 are shown in FIG. 5B, distalmost link 466 may have fewer or more articulation wire lumen(s) 490, e.g., spaced evenly or unevenly around central lumen 450. Articulation wire lumen(s) 490 may be aligned with the articulation wire lumen(s) 164 of links 160. Articulation wire lumens 490 are shown in broken lines in FIG. 5B, for example, to illustrate that articulation wire lumens 490 extend longitudinally through wall 482.

As shown in FIG. 5B, a first channel 404A and a second channel 404B may extend through wall 482 of distalmost link 466. Except as specified herein, first channel 404A may have any of the features of first channel 304A, and second channel 404B may have any of the features of second channel 304B. Portions of channels 404A, 404B, may be approximately perpendicular to a longitudinal axis of distalmost link 466 and other portions of channels 404A, 404B may be approximately parallel to longitudinal axis of distalmost link 466. First channel 404A and second channel 404B may be on opposite sides of distalmost link 466. First channel 404A may have the same features as second channel 404B. In these aspects, element numbers for features of first channel 404A may be used to identify similar element numbers for features of second channel 404B.

Channels 404A, 404B may each have a plurality of segments. In aspects, some segments of each channel 404A, 404B may be perpendicular to the longitudinal axis of distalmost link 466, and other segments of each channel 404A, 404B may be parallel to the longitudinal axis of distalmost link 466. For example, channels 404A, 404B may each include a first external cutout 403 and a second external cutout 405 on outer surface 480 of distalmost link 466. Cutouts 403 and 405 may have any of the properties of cutouts 303 and 305 of FIGS. 4A and 4B. First cutout 403 and second cutout 405 may be aligned with one another. For example, a curve (e.g., a circumference) extending circumferentially about outer surface 480 of distal link 466 and perpendicularly to the longitudinal axis of distalmost link 466 may extend along a longitudinal axis of each of first cutout 403 and second cutout 405.

Channels 404A, 404B may also define an internal cutout 407 on internal surface 478 of distalmost link 466. Internal cutout 407 may be proximal of external cutouts 403 and 405. Internal cutout 405 may have a longitudinal axis that extends along a circumference of outer surface 480. Internal cutout 407 may be circumferentially between first external cutout 403 and second external cutout 405. In aspects, internal cutout 407 may be U-shaped such that at least a first portion of internal cutout 407 extends parallel to the longitudinal axis of distalmost link 466, and at least a second portion of internal cutout 407 extend perpendicularly to the longitudinal axis of distalmost link 466. Internal cutout 407 may be a recess that is recessed with respect to inner surface 478 but does not extend all the way through wall 482. When viewed from the side as in FIG. 5A, at least a portion of internal cutout 407 may be approximately parallel to external cutouts 403 and 405. Although internal cutout 407 is disposed on inner surface 478, internal cutout 407 is shown in broken lines in FIG. 5A, for example, to illustrate a position of internal cutout 407 on inner surface 478 and relative to external cutouts 403, 405.

Shown in FIG. 5B, a first recess or lumen 406 may extend between an end of first external cutout 403 and a first end of internal cutout 407. A second recess or lumen 408 may extend between an end of second external cutout 405 and a second end of internal cutout 407. Each of lumen 406, 408 may extend approximately perpendicular to a central longitudinal axis of distalmost link 466.

A single articulation wire 412 (or group of wires such as a cable) may extend through channels 404A. For example, a first distal opening 492A of a first of articulation wire lumen(s) 490 may be disposed on a first proximal wall 488A of each of first channel 404A and second channel 404B. A second distal opening 492B of a second of articulation wire lumen(s) 490 may be disposed on a second proximal wall 488B of each of first channel 404A and second channel 404B. In aspects, first distal opening 492A and second distal opening 492B of two of the articulation wire lumens 490 may be disposed on either end of each channel 404A, 404B.

A first segment 412A, a first bend 413A, a second segment 412B, a second bend 413B, a third segment 412C, a third bend 413C, and a fourth segment 412D of articulation wire(s) 412 may be similar to respective segments and bends of articulation wire(s) 312 of FIGS. 4A and 4B.

On an opposite end of fourth segment 412D from bend 413C, articulation wire 412 may be subjected to a fourth bend 413D. Fourth bend 413D may be approximately 90 degrees in a proximal direction. After fourth bend 413D, a fifth segment 412E of articulation wire 412 may extend longitudinally in a proximal direction (e.g., parallel to the longitudinal axis of distalmost link 466) through lumen 406 and into/along internal cutout 407. On an opposite end of fifth segment 412E from fourth bend 413D, articulation wire 412 may be subjected to a fifth bend 413E. Fifth bend 413E may be approximately 90 degrees such that a sixth segment 412F extends in a direction that is perpendicular to the longitudinal axis of distalmost link 466. In aspects, sixth segment 412F may be parallel to third segment 412C.

A sixth bend 413F on an opposite end of sixth segment 412F from bend 413F may be approximately 90 degrees such that a seventh segment 412G extends in a direction that is parallel to the longitudinal axis of distalmost link 466, through lumen 408. In these aspects, seventh segment 412G may be parallel to fifth segment 412E. A seventh bend 413G may be approximately 90 degrees such that an eighth segment 412H extends radially outward, e.g., towards external surface 480 of distalmost link 466.

Articulation wire 412 may be subjected to an eighth bend 413H such that a ninth segment 412J of articulation wire 412 extends along a length of second cutout 405. On an opposite end of ninth segment 412J from bend 413H, articulation wire 412 may be subjected to a ninth bend 413J such that a tenth segment 412K of articulation wire 412 extends towards central lumen 450. On an opposite end of tenth segment 412K from bend 413J, articulation wire 412 may be subjected to a tenth bend 413K. Tenth bend 413K may be approximately 90 degrees such that an eleventh segment 412L (FIG. 5A) of articulation wire 412 extends into a second articulation wire lumen 490 of distalmost link 466 (e.g., via opening 492B disposed on second proximal wall 488B of first channel 404B). In these aspects, a longitudinal axis of eleventh segment 412L may be perpendicular to a longitudinal axis of tenth segment 412K. In these aspects, the longitudinal axis of eleventh segment 412L may be parallel to the longitudinal axis of first segment 412A and the longitudinal axis of distalmost link 466.

Bends 413H, 413J, and 413K may have any of the properties and may extend through similar structures as bends 313F, 313G, and 313H, respectively. Similarly, segments 412J, 412K, and 412L may have any of the properties of segments 312G, 312H, 312J, respectively, and may extend through similar structures.

Although first channel 404A is referred to above, it will be appreciated that a second articulation wire 412 extending through second channel 404B may follow the same path as described above. In aspects, articulation wire(s) 412 may be weaved through each respective channel 404A, 404B. In these aspects, the ten bends 413A, 413B, 413C, 413D, 413E, 413F, 413G, 413H, 413J, 413K, of distalmost link 466 may create a capstan effect on articulation wire 412, e.g., similar to the capstan effect described with regards to FIGS. 4A and 4B. Thus, articulation wire 412 may be secured within distalmost link 466. The number of bends of distalmost link 466 is not limited, however. For example, each channel 404A, 404B, may include additional segments, thus imparting additional bends on articulation wire 412 and increasing a capstan effect of articulation wire 412.

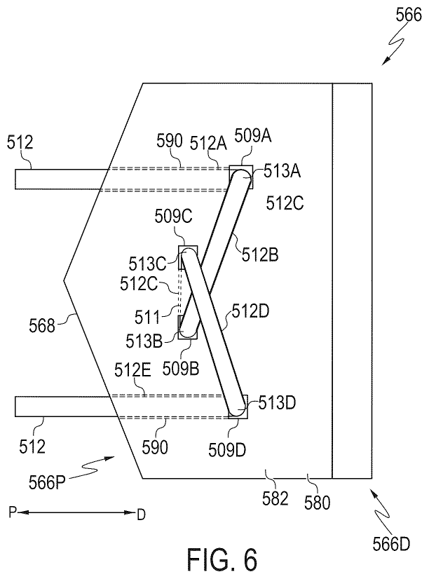

Referring to FIG. 6, an articulation wire 512 may be subjected to bends (similar to the previous embodiments) and may include portions that overlap, or criss-cross over themselves. For example, FIG. 6 illustrates a top view of an alternative distalmost link 566 having an outer surface 580. Distalmost link 566 may have any or all of the characteristics of one or more of distalmost links 166, 266, 366, 466 except as described below. For example, distalmost link 566 may be arranged along a longitudinal axis of articulation joint assembly 128. Links 160 may be arranged proximally to distalmost link 566. Distalmost link 566 may define a distalmost portion of articulation joint assembly 128. Articulation wire(s) 512 may extend through distalmost link 566, as described in further detail below. Articulation wire(s) 512 may have any or all of the characteristics of articulation wire(s) 112, 212, 312, 412 described above.

Although not shown, a central lumen may extend through distalmost link 566. A wall 582 may be defined between an inner surface and an outer surface 580 of distalmost link 566. For example, outer surface 580 may define a radially outer surface of distalmost link 566. Distalmost link 566 may have a proximal end 566P and a distal end 566D. In some aspects, proximal end 566P may be defined by one or more surfaces 568. The one or more surfaces 568 may be angled.

One or more articulation wire lumens 590 may extend at least partially through wall 582 of distalmost link 566. Articulation wire lumen(s) 590 of distalmost link 566 may extend at least partially through wall 582, e.g., parallel to the longitudinal axis of distalmost link 566. Distalmost link 566 may have two or more articulation wire lumen(s) 590 spaced evenly or unevenly around the central lumen of distalmost link 566. Articulation wire lumen(s) 590 may be aligned with the articulation wire lumen(s) 164 of links 160. Articulation wire lumens 590 are shown in broken lines in FIG. 6, for example, to illustrate that articulation wire lumens 590 extend longitudinally through wall 582 of distalmost link 566.

A first segment 512A of a single articulation wire 512 (or group of wires such as a cable) may extend through a first of articulation wire lumens 590. A second segment 512B may extend radially outward, for example, through a first window 509A (also referred to herein as a first cutout) disposed on outer surface 580. A first bend 513A may be between first segment 512A and second segment 512B.

Second segment 512B may extend along outer surface 580, for example, transverse (e.g., at a non-zero and non-right angle) to a longitudinal axis of distalmost link 566. In aspects, a first end (an end closer to first window 509A) of second segment 512B may be distal of a second end (an end opposite the first end) of second segment 512B. In other aspects, the first end of second segment 512B may be proximal of a second end of second segment 512B.

The second end of second segment 512B may extend radially inward, for example, through a second window 509B (also referred to herein as a second cutout) disposed on outer surface 580. Second window 509B may be proximal of first window 509A. A second bend 513B may be between second segment 512B and a third segment 512C. Third segment 512C may extend through a channel 511 of distalmost link 566. Channel 511 may be approximately perpendicular to a longitudinal axis of distalmost link 566. Channel 511 may be entirely disposed within wall 582 of distalmost link 566. For example, channel 511 may be a closed channel (e.g., not in fluid communication with the central lumen of distalmost link 566) or channel 511 may be disposed on an inner surface of wall 582 of distalmost link 566 such that channel 511 is exposed to (e.g., in fluid communication with) the central lumen of distalmost link 566.

Third segment 512C may extend between second window 509B and a third window 509C, for example, through channel 511. Third window 509B may be longitudinally aligned with second window 509B and proximal of first window 509A. A third bend 513C may be between third segment 512C and a fourth segment 512D.

Fourth segment 512D may extend radially outward, for example, through third window 509C (also referred to herein as a third cutout) disposed on outer surface 580. Fourth segment 512D may extend between third window 509C and a fourth window 509D. Fourth window 509D may be longitudinally aligned with first window 509A and distal to second window 509B and third window 509C. In aspects, fourth segment 512D may extend along outer surface 580 and transverse (e.g., at a non-zero and non-right angle) to a longitudinal axis of distalmost link 566. For example, a first end (and end closer to third window 509C) of fourth segment 512D may be proximal of a second end (an end closer to fourth window 509D) of fourth segment 512B. In other aspects, the first end of fourth segment 512D may be distal to a second end of fourth segment 512D. Fourth segment 512D may extend, or cross, over second segment 512B. Alternatively, second segment 512B may extend, or cross, over fourth segment 512D. A fourth bend 513D may be between fourth segment 512D and a fifth segment 512E.

The second end of fourth segment 512D may extend radially inward, for example, through a fourth window 509D (also referred to herein as a fourth cutout) disposed on outer surface 580. Fifth segment 512E may extend through a second of articulation wire lumens 590.

Although second window 509B and third window 509C are depicted as being proximal of first window 509A and fourth window 509D, such a configuration is merely exemplary. In alternatives, second window 509B and third window 509C may be distal to first window 509A and fourth window 509D.

In some aspects, outer surface 580 may include one or more grooves, for example, configured to receive second segment 512B and/or fourth segment 512D. Similarly, the inner surface of distalmost link 566 may include one or more grooves, for example, configured to receive third segment 512C. The grooves may assist in maintaining a position of second segment 512B, third segment 512C, and/or fourth segment 512D.

In further aspects, second segment 512B and fourth segment 512D may extend along an inner surface of distalmost link 566 (e.g., along an inner surface that defines the central lumen of distalmost link 566), and third segment 512C may extend along outer surface 580 of distalmost link 566.

Articulation wire 512 may include additional segments and/or bends, for example, between each of the described segments of articulation wire 512 (e.g., first segment 512A, second segment 512B, third segment 512C, fourth segment 512D, and/or fifth segment 512D). The additional segments/bends on articulation wire 512 may increase a capstan effect of articulation wire 512.

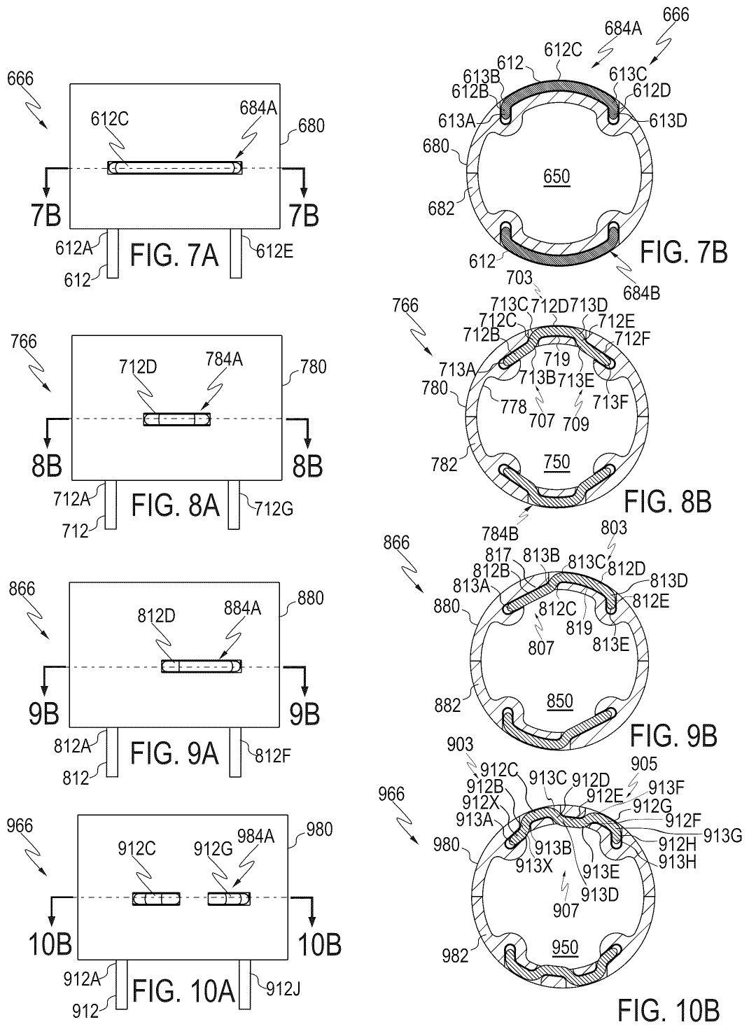

FIGS. 7A-10B illustrate various distalmost links and respective cross-sectional views. For example, FIG. 7A illustrates a top view of a distalmost link 666, and FIG. 7B illustrates a cross-sectional view of distalmost link 666. FIG. 8A illustrates a top view of a distalmost link 766, and FIG. 8B illustrates a cross-sectional view of distalmost link 766. FIG. 9A illustrates a top view of a distalmost link 866, and FIG. 9B illustrates a cross-sectional view of distalmost link 866. FIG. 10A illustrates a top view of distalmost link 966, and FIG. 10B illustrates a cross-sectional view of distalmost link 966.

Referring to FIGS. 7A and 7B, distalmost link 666 may have any or all of the characteristics of distalmost links 166, 266, 366, 466, and/or 566, described above, except as described below. For example, a central lumen 650 may extend through distalmost link 666. A wall 682 of distalmost link 666 may include a first cutout 684A and a second cutout 684B (FIG. 7B), each of which may extend at least partially into a wall 682 of distalmost link 666 from an outer surface 680 of link 666. Cutouts 684A, 684B may each be mirror images of one another, and, thus, may each have the same features. An articulation wire 612 may extend through articulation wire lumens of wall 682 (e.g., via one or more articulation wire lumens similar to articulation wire lumen(s) 190, 290, 390, 490, 590, described above). Articulation wire 612 may also extend through cutouts 684A, 684B. Articulation wire 612 may have any or all of the characteristics of articulation wire 112, 212, 312, 412, 512, described above.

In aspects, articulation wire 612 may include a plurality of segments and a plurality of bends. For simplicity, the plurality of segments and bends are called out for articulation wire 612 extending through first cutout 684A. However, articulation wire 612 extending through second cutout 684B may have any or all of the same bends and segments. For example, articulation wire 612 may include a first segment 612A, a first bend 613A, a second segment 612B, a second bend 613B, a third segment 612C, a third bend 613C, a fourth segment 612D, a fourth bend 613D, and a fifth segment 612E. Each segment and bend may have any or all of the same characteristics the segments and bends previously described with respect to FIGS. 2A, 2B, 3, 4A, 4B, 5A, 5B, 6. Third segment 612C may extend partially around an outer circumference of distalmost link 666, for example, within cutout 684A, 684B. In aspects, articulation wire 612 may not be exposed to central lumen 650. For example, distalmost link 666 may not include any cutouts that are in communication with central lumen 650.

Referring to FIGS. 8A and 8B, distalmost link 766 may have any or all of the characteristics of distalmost link 166, 266, 366, 466, 566, and/or 666, described above, except as described below. For example, a central lumen 750 may extend through distalmost link 766. A wall 782 of distalmost link 766 may include a first channel 784A and a second channel 784B (FIG. 8B), each of which may extend at least partially into a wall 782 of distalmost link 766 from an outer surface 780 of link 766. Channels 784A, 784B may each be mirror images of one another, and, thus, may each have the same features. In aspects, each of first channel 784A a second channel 784B may define one or more internal or external cutouts. For example, first channel 784A and second channel 784B may define a first internal cutout 707 (also referred to herein as a first internal window), a first external window 703 (also referred to herein as a first external window), and a second internal cutout 709 (also referred to herein as a second internal window).

An articulation wire 712 may extend through articulation wire lumens of wall 782 (e.g., via one or more articulation wire lumens similar to articulation wire lumen(s) 190, 290, 390, 490, 590, described above). Articulation wire 712 may also extend through channels 784A, 784B. Articulation wire 712 may have any or all of the characteristics of articulation wire 112, 212, 312, 412, 512, 612 described above.

In aspects, articulation wire 712 may include a plurality of segments and a plurality of bends. For simplicity, the plurality of segments and bends are called out for articulation wire 712 extending through first channel 784A. However, articulation wire 712 extending through second channel 784B may have any or all of the same bends and segments. For example, articulation wire 712 may include a first segment 712A, a first bend 713A, a second segment 712B, a second bend 713B, a third segment 712C, a third bend 713C, a fourth segment 712D, a fourth bend 713D, a fifth segment 712E, a fifth bend 713E, a sixth segment 712F, a sixth bend 713F, and a seventh segment 712G. Each segment and bend may have any or all of the same characteristics the segments and bends previously described with respect to FIGS. 2A, 2B, 3, 4A, 4B, 5A, 5B, 6, 7A, 7B.

Each of second segment 712B and sixth segment 712F may extend through a portion of channel 784A that is exposed to central lumen 750 (e.g., recessed into wall 782 from an inner surface 778 of link 766). For example, second segment 712B may extend through first internal cutout 707, and sixth segment 712F may extend through second internal cutout 709. First internal cutout 707 and second internal cutout 709 may extend partially around an inner circumference of distalmost link 766, for example, along inner surface 778. Fourth segment 712D may partially extend around an outer circumference of distalmost link 766, for example, within external cutout 703. For example, fourth segment 712D may be exposed to an environment surrounding distalmost link 766. A radially inner surface 719 may also assist in forming fourth segment 712D, third bend 713C, and fourth bend 713D.

Referring to FIGS. 9A and 9B, distalmost link 866 may have any or all of the characteristics of distalmost link 166, 266, 366, 466, 566, 666, and 766, described above, except as described below. For example, a central lumen 850 may extend through distalmost link 866. A wall 882 of distalmost link 866 may include a first channel 884A and a second channel 884B (FIG. 9B), each of which may extend at least partially into a wall 882 of distalmost link 866 from outer surface 880. Channels 884A, 884B may each be mirror images of one another, and, thus, may each have the same features. In some aspects, a shown, channels 884A, 884B may each be reverse mirror images of one another. Channels 884A, 6884B may each define a first internal cutout 807 and a first external cutout 803.

An articulation wire 812 may extend through articulation wire lumens of wall 882 (e.g., via one or more articulation wire lumens similar to articulation wire lumen(s) 190, 290, 390, 490, 590, described above). Articulation wire 812 may also extend through channels 884A, 884B. Articulation wire 812 may have any or all of the characteristics of articulation wire 112, 212, 312, 412, 512, 612, 712 described above.

In aspects, articulation wire 812 may include a plurality of segments and a plurality of bends. For simplicity, the plurality of segments and bends are called out for articulation wire 812 extending through first channel 884A. However, articulation wire 812 extending through second channel 884B may have any or all of the same bends and segments. For example, articulation wire 812 may include a first segment 812A, a first bend 813A, a second segment 812B, a second bend 813B, a third segment 812C, a third bend 813C, a fourth segment 812D, a fourth bend 813D, a fifth segment 812E, a fifth bend 813E, and a sixth segment 812F. Each segment and bend may have any or all of the same characteristics the segments and bends previously described with respect to FIGS. 2A, 2B, 3, 4A, 4B, 5A, 5B, 6, 7A, 7B, 8A, 8B.

Second segment 812B may extend through first internal cutout 807 of channel 884A. For example, second segment 812B may extend partially around an inner circumference of distalmost link 866, for example, within first internal cutout 807 of channel 884A. Fourth segment 812D may partially extend around an outer circumference of distalmost link 66, for example, through external cutout 803 of channel 884A. For example, fourth segment 812D may be exposed to an environment surrounding distalmost link 866.

A radially outer surface 817 may assist in forming second segment 812B, second bend 813B, and third segment 812C. A radially inner surface 819 may also assist in forming second bend 813B and third segment 812C, in addition to third bend 813C, fourth segment 812D, and fourth bend 813D. In aspects, first internal cutout 807 and/or first external cutout 803 may be off-center with respect to a center point between first segment 812A and sixth segment 812F.

Referring to FIGS. 10A and 10B, distalmost link 966 may have any or all of the characteristics of distalmost link 166, 266, 366, 466, 566, 666, 766, and 866 described above, except as described below. In particular, distalmost link 966 may have any or all of the characteristics of distalmost link 366, except as described below. For example, distalmost link 966 may impart a similar “W” or “M” shape to articulation wire 912.

A central lumen 950 may extend through distalmost link 966. A wall 982 of distalmost link 966 may include a first channel 984A and a second channel 984B (FIG. 10B), each of which may extend at least partially into a wall 982 of distalmost link 966. Channels 984A, 984B may each be mirror images of one another, and, thus, may each have the same features. In some aspects, channels 984A, 984B may each be reverse mirror images of one another. Articulation wire 912 may extend through articulation wire lumens of wall 982 (e.g., via one or more articulation wire lumens similar to articulation wire lumen(s) 190, 290, 390, 490, 590, described above). Articulation wire 912 may also extend through channels 984A, 984B. Articulation wire 912 may have any or all of the characteristics of articulation wire 112, 212, 312, 412, 512, 612, 712, 812 described above.

In aspects, articulation wire 912 may include a plurality of segments and a plurality of bends. For simplicity, the plurality of segments and bends are called out for articulation wire 912 extending through first channel 984A. However, articulation wire 912 extending through second channel 984B may have any or all of the same bends and segments. In aspects, distalmost link 966 may be similar to distalmost link 366. Thus, articulation wire 912 may include a same or similar number of segments and bends. However, articulation wire 912 may include an additional segment 912X and an additional bend 913X, for example, between first segment 912A and second segment 912B. Additional segment 912X may extend through a portion of wall 982, for example, perpendicular to a longitudinal axis of distal most link 966.

For example, articulation wire 912 may include a first segment 912A, a first bend 913A, an additional segment 912X, an additional bend 913X, a second segment 912B, a second bend 913B, a third segment 912C, a third bend 913C, a fourth segment 912D, a fourth bend 913D, a fifth segment 912E, a fifth bend 913E, a sixth segment 912F, a sixth bend 913F, a seventh segment 912G, a seventh bend 913F, an eighth segment 912H, an eighth bend 913H, and a ninth segment 912J. Each segment and bend may have any or all of the same characteristics the segments and bends previously described with respect to FIGS. 2A, 2B, 3, 4A, 4B, 5A, 5B, 6, 7A, 7B, 8A, 8B, particularly of FIGS. 4A and 4B. The additional segment 912X and additional bend 913X may further improve, or add to, a capstan effect on articulation wire 912.

Distalmost link 966 may include a first external cutout 903, a first internal cutout 907, and a second external cutout 905. Each of cutouts 903, 905, and 907 may have any or all of the same characteristics of cutouts 303, 305, and 307 of FIGS. 4A and 4B. For example, one or more of cutouts 903, 905, and/or 907, may be off-centered with respect to a center point between first segment 912A and ninth segment 912J.

Each of the segments and bends discussed above with respect to FIGS. 7A-10B may create a capstan effect on each respective articulation wire. For example, a tensile force applied to articulation wire 612, 712, 812, and/or 912 (e.g., during articulation) may increase exponentially due to the plurality of bends imparted on the respective articulation wire. In these aspects, the bends may assist in preventing the articulation wire from being pulled proximally out of the respective distalmost link.

Furthermore, aspects of each of distalmost links 166, 266, 366, 466, 566, 666, 766, 866, 966 may be used in combination with one another. For example, a first half of a distalmost link may have characteristics of one of the above-listed distalmost links, and the other half of the distalmost link may have characteristics of another of the above-listed distalmost links. In further aspects, of each of the above-listed distalmost links may be used in combination to increase a number of segments and bends imparted on an articulation wire. For example, a distalmost link may include additional cutouts, surfaces, and/or channels and improve a capstan effect on the articulation wire.

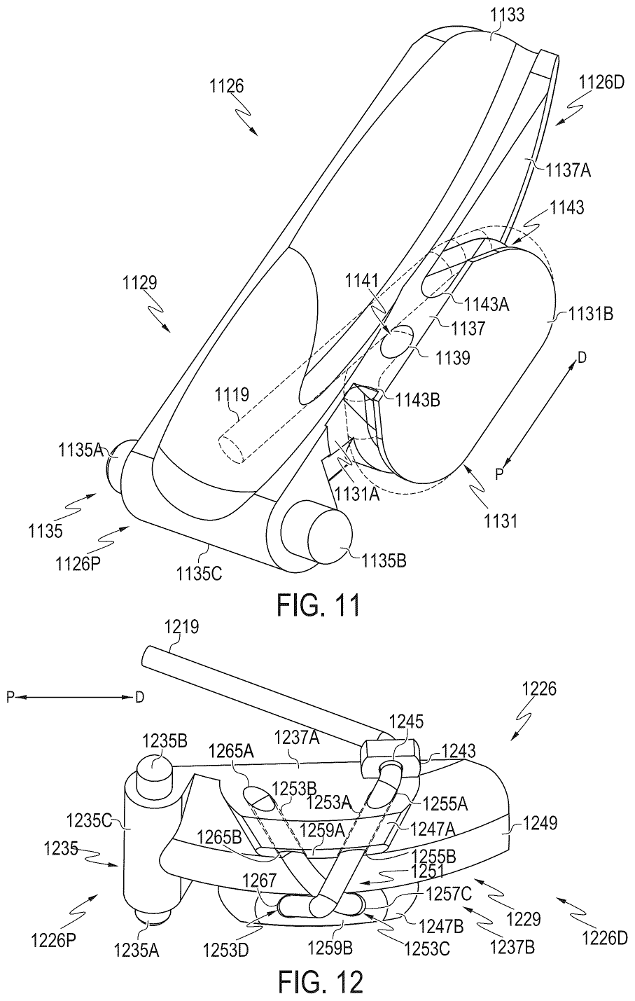

FIGS. 11-12 illustrate exemplary elevators and features thereof. Although features may be described with respect to a particular example herein, it will be appreciated that the features described herein may be combined in any suitable combination. Either of the elevators described herein may be used in conjunction with duodenoscope 10 or another medical device and may have any of the features of elevator 26.

FIG. 11 illustrates a perspective view of an example elevator 1126. Elevator 1126 may include a body 1129 and a coupler 1131 for fixing an elevator wire 1119 to elevator 1126. Body 1129 may have one or more guide surfaces 1133. Guide surfaces 1133 may have a “U” or “V” shape. An axle 1135 may be at a proximal end 1126P of elevator 1126 (e.g., opposite a distal end 1126D of elevator 1126). Axle 1135 may be coupled to or integrally formed with body 1129. In aspects, a diameter of axle 1135 on a first end 1135A and/or a second end 1135B may be smaller than a diameter of a center portion 1135C of axle 1135 (i.e., center portion 1135C may be between first end 1135A and second end 1135B). One or both of first end 1135A and/or second end 1135B may be received within a portion of distal tip 20 (see FIG. 1A), for example, to movably (e.g., rotatably) fix elevator 1126 within distal tip 20. Elevator 1126 may be formed by any suitable method of manufacturing, including additive or subtractive manufacturing methods such as molding, three-dimensional printing, carving, or other methods.

Coupler 1131 may extend laterally away from a longitudinal axis of elevator 1126. For example, coupler 1131 may extend outward from a side surface 1137A of body 1129. In aspects, coupler 1131 may resemble a spool (e.g., an oblong spool). For example, a first wall 1131A of coupler 1131 may be integrally formed with side surface 1137A of body 1129 and a second wall 1131B may be on an opposite side of a groove 1143. First wall 1131A may be coupled to or formed with side surface 1137A of body 1129. Groove 1143 may extend circumferentially around at least a portion of coupler 1131. Groove 1143 may be sized and shaped to receive an elevator wire 1119. Elevator wire 1119 is shown in broken lines to more clearly illustrate aspects of elevator 1126.

Coupler 1131 may further include a planar surface 1137 on a front side of elevator 1126 (e.g., a side of elevator 1126 having guide surface 1133). In aspects, planar surface 1137 may extend between first wall 1131A and second wall 1131B and longitudinally between a first groove opening 1143A and a second groove opening 1143B of groove 1143. First groove opening 1143A and a second groove opening 1143B may be disposed on planar surface 1137. First groove opening 1143A may be an opening to a first end of groove 1143, and second groove opening 1143B may be an opening to a second end of groove 1143. An opening 1139 of a cavity 1141 may be disposed on planar surface 1137, for example, between first groove opening 1143A and second groove opening 1143B. Cavity 1141 may be a thru hole extending through an entirety of coupler 1131 or a blind hole extending only through a portion of coupler 1131. Cavity 1141 may be sized and shaped to receive an end of elevator wire 1119.

In aspects, elevator wire 1119 may be disposed within groove 1143 of coupler 1131. For example, elevator wire 1119 may extend from a distal end of shaft 18 (FIG. 1A), through first groove opening 1143A and into groove 1143. Elevator wire 1119 may then be looped around coupler 1131 at least once such that an end portion of elevator wire 1119 extends out of groove 1143 via second groove opening 1143B on planar surface 1137. An end of elevator wire 1119 may then extend into cavity 1141 via opening 1139 on planar surface 1137. For example, the end of elevator wire 1119 may extend from the front towards the back of elevator 1126. The distalmost end of elevator wire 1119 may be secured within cavity 1141 various ways. For example, elevator wire 1119 may be secured within cavity 1141 using an epoxy or adhesive and/or cavity 1141 may be heat staked. In these aspects, elevator wire 1119 may be secured to elevator 1126.

Although not shown, elevator wire 1119 may be looped around coupler 1131 and within groove 1143 more than once. For example, elevator wire 1119 may be looped around groove 1143 multiple times. In aspects, coupler 1131 may include additional grooves 1143 to facilitate additional loops of elevator wire 1119 around coupler 1131. For example, a first loop may be around a first groove and a second loop may be around a second groove.

FIG. 12 illustrates a back perspective view of an example elevator 1226. Elevator 1226 may have any or all of the characteristics of elevator 1126, except as described below. For example, elevator 1226 may include a body 1229. Although not shown, elevator 1226 may have one or more guide surfaces on a front side of body 1229. The guide surfaces of elevator 1226 may have a “U” or “V” shape, similar to guide surfaces 1133 shown in FIG. 11. An axle 1235 may be at a proximal end 1226P of elevator 1226 (e.g., opposite a distal end 1226D). Axle 1235 may be coupled to or formed with body 1229. In aspects, a diameter of axle 1235 on a first end 1235A and/or a second end 1235B may be smaller than a diameter of a center portion 1235C of axle 1235 (e.g., between first end 1235A and second end 1235B). One or both of first end 1235A and/or second end 1235B may be received within a portion of distal tip 20 (see FIG. 1A), for example, to movably fix elevator 1226 within distal tip 20. Elevator 1226 may be formed by any suitable method of manufacturing, including additive or subtractive manufacturing methods such as molding, three-dimensional printing, carving, or other methods.

Elevator 1226 may include a projection 1243 extending outward from a first side surface 1237A of elevator 1226. An aperture 1245 may extend through an entirety of projection 1243. Aperture 1245 may extend from a front of elevator 1226 (i.e., the side of elevator 1226 having the guide surfaces) to a back of elevator 1226 (i.e., an opposite side of elevator 1226) Aperture 1245 may be sized and shaped to receive an elevator wire 1219. Elevator wire 1219 may have any or all of the characteristics of elevator wire 1219, previously discussed. Portions of elevator wire 1219 are shown in broken lines when within portions of elevator 1226.

Elevator 1226 may further include a first protrusion 1247A and a second protrusion 1247B, each of which may extend outward (back) from a back surface 1249 of elevator 1226. First protrusion 1247A and second protrusion 1247B may resemble wings on back surface 1249 of elevator 1226. A space 1251 may be defined between each protrusion 1247A, 1247B.

Each of first protrusion 1247A and second protrusion 1247B may include multiple thru holes extending through an entirety of each protrusion 1247A, 1247B. For example, first protrusion 1247A may include a first thru hole 1253A and a second thru hole 1253B. Second protrusion 1247B may include a third thru hole 1253C and a fourth thru hole 1253D. Each thru hole 1253A, 1253B, 1253C, 1253D may be sized and shaped to receive elevator wire 1219.

Each thru hole 1253A, 1253B, 1253C, 1253D may be angled. Both of first thru hole 1253A of first protrusion 1247A and third thru hole 1253C of second protrusion 1247B may be angled proximally moving in a radially inward direction (i.e., moving toward a central longitudinal axis of elevator 1222). For example, a first opening 1255A of first thru hole 1253A may be disposed on a first side surface 1237A of elevator 1226. First opening 1255A may be more distal to a second opening 1255B of first thru hole 1253A disposed on an inside surface 1259A of first protrusion 1247A. Although not shown, a first opening of third thru hole 1253C may be disposed on a second side surface of elevator 1226 (i.e., a side surface of elevator 1226 facing into the page). First opening of third thru hole 1253C may be more distal to a second opening 1257C of third thru hole 1253C disposed on an inside surface 1259B of second protrusion 1247B.

Second thru hole 1253B of first protrusion 1247A and fourth thru hole 1253D of second protrusion 1247B may be angled distally, moving in a radially inward direction (i.e., moving toward a central longitudinal axis of elevator 1222). For example, a first opening 1265A of second thru hole 1253B may be disposed on first side surface 1237A of elevator 1226. First opening 1265A of second thru hole 1253B may be more proximal to a second opening 1265B of second thru hole 1253B disposed on inside surface 1259A of first protrusion 1247A. Similarly, although not shown, a first opening of fourth thru hole 1253D of second protrusion 1247B may be disposed on the second side surface of elevator 1226 (i.e., a side surface of elevator 1226 facing into the page). A second opening 1267 of fourth thru hole 1253D of second protrusion 1247B may be disposed on inside surface 1259B of second protrusion 1247B. The first opening (not shown) of fourth thru hole 1253D may be proximal to second opening 1267 of fourth thru hole 1253D.

Elevator wire 1219 may extend through projection 1243, e.g., via aperture 1245. For example, elevator wire 1219 may extend from a front of elevator 1226 toward a back of elevator 1226. Elevator wire 1219 may then extend through first thru hole 1253A of first protrusion 1247A. Elevator wire 1219 may then extend diagonally across space 1251 and through fourth thru hole 1253D of second protrusion 1247B. Elevator wire 1219 may extend along the second side surface of elevator 1226 (e.g., the side surface of elevator 1226 facing into the page) and extend through third thru hole 1253C of second protrusion 1247B. Elevator wire 1219 may extend diagonally across space 1251 again, e.g., under or over a portion of the elevator wire 1219 extending across space 1251 and between first thru hole 1253A and fourth thru hole 1253D. An end of elevator wire 1219 may be fixed within second thru hole 1253B of first protrusion 1247A. For example, the distal end of elevator wire 1219 may be secured within second thru hole 1253B of first protrusion 1247A using a glue or adhesive, and/or by heat staking the actuation wire within second thru hole 1253B. In aspects, elevator wire 1219 may be secured within first thru hole 1253A, third thru hole 1253C, and/or fourth thru hole 1253D using same or similar means (e.g., adhesive and/or heat staking techniques).

In some aspects, elevator wire 1219 may resemble and perform similar to a rope around a nautical cleat. For example, as elevator wire 1219 is tensioned (e.g., during actuation of elevator 1226), the overlapping portion of elevator wire 1219 may tighten against a back surface 1249 of elevator 1226. Elevator wire 1219 may criss-cross, cross over itself, or at least partially overlap, along a back side of elevator 1226 (e.g., within space 1251 between first protrusion 1247A and second protrusion 1247B). In these aspects, elevator wire 1219 may be secured to elevator 1226.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented embodiments can be combined to achieve a same or similar result as one or more of the disclosed embodiments. Accordingly, the invention is not to be considered as limited by the foregoing description.

We claim:

1. An articulation portion of a medical device, comprising:
a plurality of links arranged longitudinally about an axis, wherein each link includes a center lumen and at least one lumen extending longitudinally through a wall of each respective link, wherein the at least one lumen of each link is configured to receive an articulation wire, and wherein a distalmost link of the plurality of links includes:
a first cutout on first radial surface of the distalmost link;
a second cutout on a second radial surface of the distalmost; and
a third cutout on the first radial surface of the distalmost link,
wherein the first radial surface is one of a radially outer surface or a radially inner surface, and the second radial surface is another of the radially outer surface or the radially inner surface, and wherein the articulation wire extends through each of the first cutout, the second cutout, and the third cutout.

2. The articulation portion of claim 1, wherein the first cutout is configured to impart a first bend on the articulation wire.

3. The articulation portion of claim 2, wherein the third cutout is configured to impart a second bend on the articulation wire.

4. The articulation portion of claim 3, wherein the second cutout is configured to impart a third bend on the articulation wire.

5. The articulation portion of claim 4, wherein each of the first bend, the second bend, and the third bend is approximately 180 degrees.

6. The articulation portion of claim 1, wherein each of the first cutout and the second cutout extends only partially through a wall of the distalmost link.

7. The articulation portion of claim 1, wherein the distalmost link further includes a first articulation wire lumen extending longitudinally through a wall defining the distalmost link, wherein the first articulation wire lumen is in fluid connection with the first cutout, and wherein articulation wire extends through the first articulation wire lumen.

8. The articulation portion of claim 7, wherein the distalmost link further includes a second articulation wire lumen extending longitudinally through a wall defining the distalmost link, wherein the second articulation wire lumen is in fluid connection with the second cutout, and wherein articulation wire extends through the second articulation wire lumen.

9. The articulation portion of claim 8, wherein a proximal opening of each of the first articulation wire lumen and the second articulation wire lumen is disposed on a proximal surface of the distalmost link.

10. The articulation portion of claim 9, wherein a distal opening of each of the first articulation wire lumen and the second articulation wire lumen is disposed on a proximal surface of the first cutout and the second cutout, respectively.

11. The articulation portion of claim 1, wherein the third cutout extends only partially through a wall of the distalmost link.

12. The articulation portion of claim 1, wherein the articulation wire extends from the first cutout to the third cutout via a first opening defined therebetween, and wherein the articulation wire extends from the third cutout to the second cutout via a second opening defined therebetween.

13. The articulation portion of claim 1, wherein the third cutout includes a protrusion.

14. The articulation portion of claim 13, wherein the articulation wire loops around the protrusion.

15. The articulation portion of claim 14, wherein the articulation wire overlaps itself on a proximal side of the protrusion.

16. An articulation portion of a medical device, comprising:
a plurality of links arranged longitudinally about an axis, wherein each link includes a center lumen and at least one lumen extending longitudinally through a wall of each respective link, wherein the at least one lumen of each link is configured to receive an articulation wire,
wherein a distalmost link of the plurality of links includes a channel having:
a first window disposed on a radially outer surface of the distalmost link; and
a second window disposed on a radially outer surface of the distalmost link, wherein a curve extending circumferentially about the outer surface of the distalmost link extends longitudinally through each of the first window and the second window,
wherein the articulation wire extends through each of the first window and the second window of the channel.

17. The articulation portion of claim 16, wherein the channel further includes:
a third window disposed on a radially inner surface of the distalmost link, wherein at least a portion of the third window is proximal of and parallel to the first window and the second window.

18. An elevator of a medical device, comprising:
a body; and
a coupler configured to receive an elevator wire, wherein the coupler extends laterally from a surface of the body, and wherein the elevator wire is configured to loop around the coupler.

19. The elevator of claim 18, wherein the coupler includes a cavity having an opening disposed on a front face of the coupler, wherein the cavity is configured to receive a distal end of the elevator wire.

20. The elevator of claim 19, wherein the coupler includes a groove extending around at least a portion of the coupler, wherein the groove receives the elevator wire.
